(19) United States
(12) Patent Application Publication
(10) Pub. No.: US 2024/0000465 A1
(43) Pub. Date: Jan. 4, 2024

(54) Hands Free Drill Guide

(71) Applicant: Tyber Medical LLC, Bethlehem, PA (US)

(72) Inventors: Jesse Feinberg, Emmaus, PA (US); David Hannah, Nazareth, PA (US)

(21) Appl. No.: 18/214,669

(22) Filed: Jun. 27, 2023

Related U.S. Application Data
(60) Provisional application No. 63/356,579, filed on Jun. 29, 2022.

(51) Int. Cl. A61B 17/17

(52) U.S. Cl. CPC A61B 17/1728; A61B 2017/0046

(57) ABSTRACT

A hands free drill guide includes a handle having a proximal handle end portion, a distal gripping end portion, and a longitudinal through-passage extending between the proximal end portion and the distal gripping end portion. The distal end portion includes two legs extending along either side of the through passage. A method of using the drill guide is also provided.

### CROSS-REFERENCE TO RELATED APPLICATION

The present application claims the benefit of U.S. Provisional Patent Application Ser. No. 63/356,579, filed on Jun. 29, 2022, which is incorporated herein by reference in its entirety.

### BACKGROUND OF THE INVENTION

### Field of the Invention

The invention relates to a drill guide that is used to guide a drill during implantation of a plate to repair a bone or bone fragments.

### Description of the Related Art

Drilling a bone for the insertion of screws or other devices to repair damaged or broken bones is a tedious process that often requires multiple clinicians to hold various instruments, there by cluttering up and obscuring the work site, which can lead to errors in drilling into the bone.

It would be beneficial to provide a drill guide that does not have to be held during use to free up the clinician's hands to allow a drill to be inserted through the drill guide and drill a hole in a bone for securing a plate to the bone.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a hands free drill guide includes a handle having a proximal handle end portion, a distal gripping end portion, and a longitudinal through-passage extending between the proximal end portion and the distal gripping end portion. The distal end portion includes two legs extending along either side of the through passage.

A method of using the drill guide is also provided.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of a drill guide according to an exemplary embodiment of the present invention inserted into a bone plate;

FIG. 2 is a side elevational view of the guide of FIG. 1;

FIG. 3 is a top plan view of the plate of FIG. 1;

FIG. 4 is an enlarged view of a distal end of one leg of the guide of FIG. 1, taken along circle 4;

FIG. 5 is a side elevational view, in section, of the guide of FIG. 1 being inserted into the plate of FIG. 1; and

FIG. 6 is an enlarged elevational view, in section, of the guide of FIG. 5 inserted into the plate of FIG. 5.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

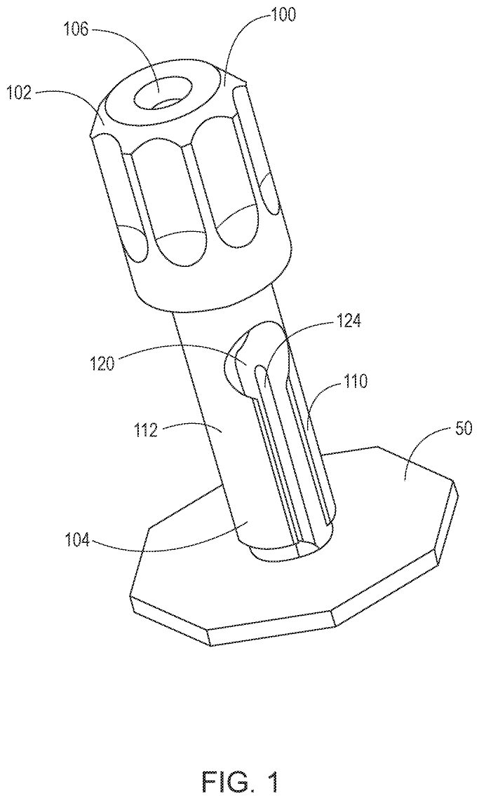

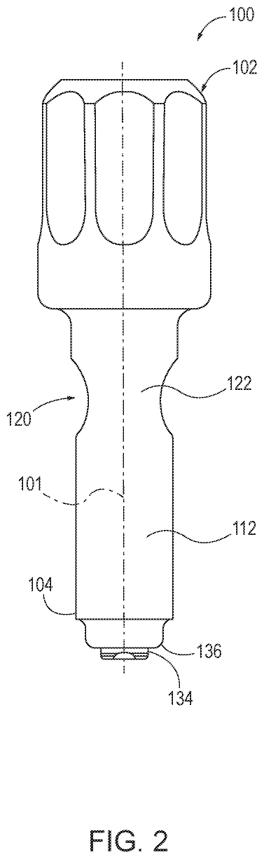

Referring to FIGS. 1, 2, and 4-6, a hands free drill guide 100 according to an exemplary embodiment of the present invention is shown. Hands-free drill guide 100 ensures accurate drilling by providing a stable and consistent drilling angle, as well as eliminates the risk of drilling at an incorrect angle, resulting in more precise drill holes.

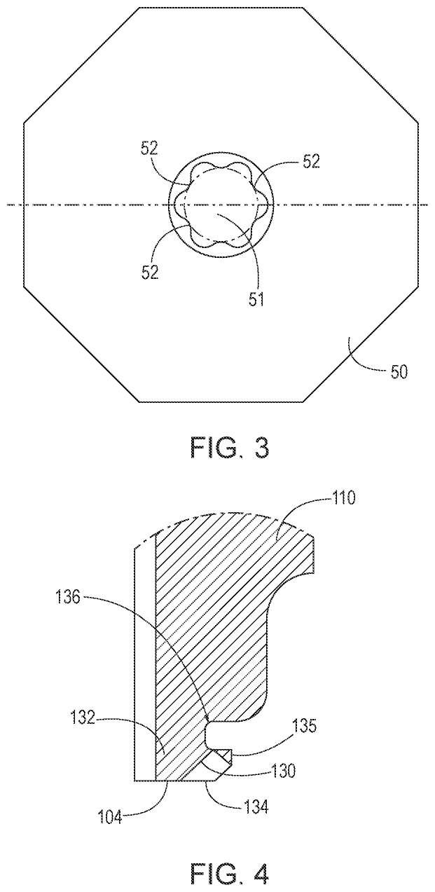

Guide 100 is used to allow a drill (not shown) to be inserted through guide 100 and drill a hole in a bone (not shown) for securing a plate 50 having an opening 51 with hex lobes 52 to the bone. Plate 50 is shown in FIGS. 1 and 3 as an octagonal plate, although those skilled in the art will recognize that plate 50 can be any shape. Handle 100 grips lobes 52 to secure handle 100 to plate 50.

Guide 100 includes a proximal handle end portion 102 extending along a longitudinal axis 101, a distal gripping end portion 104, and a longitudinal through-passage 106 extending between proximal end portion 102 and distal gripping end portion 104. Proximal handle end portion 102 includes a known contoured outer surface to provide for gripping and to enhance a clinician's ability to insert guide 100 into plate 50. In an exemplary embodiment, guide 100 is a single piece of ASTM F899 stainless steel, although those skilled in the art will recognize that guide 100 can be constructed from other materials as well.

Distal end portion 104 includes two legs 110, 112 that extend along either side of through passage 106. Legs 110, 112 are initially parallel to each other, but are biased toward each other during insertion into plate opening 51. To assist in this biasing feature, a transverse generally circular slot 120 is formed at a proximal end 122 of each of legs 110, 112. Slot 120 allows legs 110, 112 to bend toward each other at proximal ends 122.

Legs 110, 112 are mirror images of each other across a central longitudinal plane, so only leg 110 will be discussed in detail. A longitudinal trough 124 extends along an inner surface of leg 110 between slot 120 and distal gripping end portion 104 of guide 100. Trough 124 accommodates the drill when the drill is inserted through guide 100.

Referring to FIG. 4, a detail of distal gripping end portion 104 of leg 110 is shown. A tab 130 is provided at the most distal end 132 of leg 110. Tab 130 includes an outer radial chamfer 134. In an exemplary embodiment, chamfer 134 is cut at a 55 degree angle relative to most distal end 132. Chamfer 134 allows leg 110 to bias inwardly toward leg 112 when distal end portion 104 of guide 100 is inserted into plate opening 51. Chamfer 134 extends vertically at a chamfer end 135 to a radial groove 136. Groove 136 is provided to engage a lobe 52 in plate 50 to secure guide 100 to plate 50.

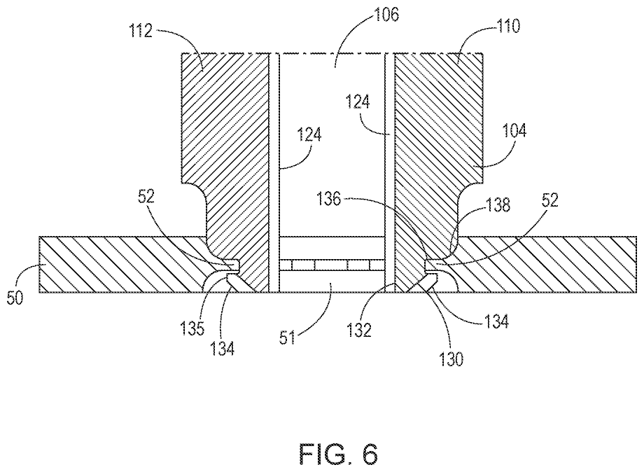

A convex radial portion 138 extends above and radially outwardly from groove 136. As shown in FIG. 6, convex radial portion 138 is sized and contoured to accommodate plate opening 51 above lobes 52.

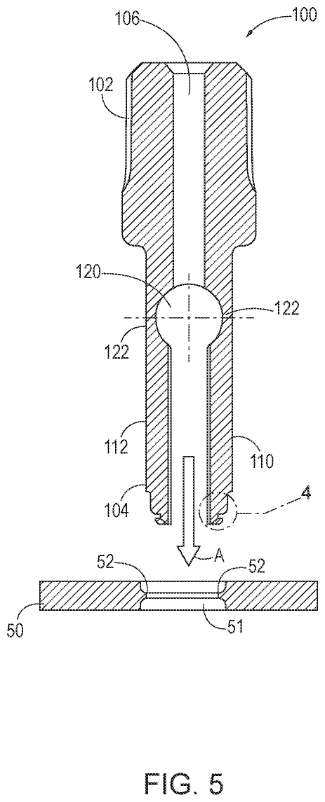

To insert guide 100 into plate 50, guide 100 is located over plate opening 51, as shown in FIG. 5. Guide 100 is inserted into plate opening 51 as indicated by arrow “A” in FIG. 5. Chamfer 134 on each of legs 110, 112 engages the top of diametrically opposing lobes 52. Each chamfer 134 rides along its respective lobe 52, biasing legs 110, 112 toward each other until the chamfer end 135 is forced inward of lobe 52. As guide 100 is further forced downward in direction “A”, groove 136 engages lobes 52, allowing legs 110, 112 to bias outwardly toward their original positions, securing legs 110, 112 into plate opening, as shown in FIG. 6.

With guide 110 now securely attached to plate 50, guide 100 can be released by the clinician. Guide 100 will remain in place, allowing the clinician a free hand to better enable the clinician to insert the drill into through passage 106 to drill into the bone.

The clinician removes the guide 100 by lightly pinching the distal end of the guide legs 110, 112 inward toward each other until groove 136 is clear of lobes 52. The guide 100 is then lifted upward out of the plate 50. Likewise, the guide 100 may be removed by angling the proximal ends 122 of leg 110,112 until groove 136 snaps out of lobes 52.

It will be further understood that various changes in the details, materials, and arrangements of the parts which have been described and illustrated in order to explain the nature of this invention may be made by those skilled in the art without departing from the scope of the invention as expressed in the following claims.

We claim:

1. A hands free drill guide comprising:
a handle having a proximal handle end portion, a distal gripping end portion, and a longitudinal through-passage extending between the proximal end portion and the distal gripping end portion,
wherein the distal end portion includes two legs extending along either side of the through passage.

2. The hands free drill guide according to claim 1, wherein the legs movable between a first position wherein the legs are parallel to each other, and a second position, wherein the legs are biased toward each other during insertion into plate a.

3. The hands free drill guide according to claim 2, wherein a transverse slot is formed at a proximal end of each of the legs, such that the slot allows the legs to bend toward each other the proximal ends.

4. The hands free drill guide according to claim 3, further comprising a longitudinal trough extending along an inner surface of each leg between the slot and a distal gripping end portion of the guide, wherein the trough is sized to accommodates a drill when the drill is inserted through the guide.

5. The hands free drill guide according to claim 4, further comprising a tab located at a distal end of each leg.

6. The hands free drill guide according to claim 5, wherein the tab includes an outer radial chamfer, wherein the chamfer allows each leg to bias inwardly toward the other leg when the distal gripping end portion of the guide is inserted into a plate opening.

7. The hands free drill guide according to claim 6, wherein the chamfer extends vertically at a chamfer end to a radial groove.

8. The hands free drill guide according to claim 7, further comprising a convex radial portion extending above and radially outwardly from the groove.

9. A method of using a drill guide, comprising the steps of:
(a) providing the drill guide according to claim 7;
(b) locating the drill guide over the plate opening; and
(c) inserting the drill guide into the plate opening.

10. The method according to claim 9, wherein step (c) comprises engaging a top of diametrically opposing lobes with the chamfer on each of the legs.

11. The method according to claim 10, wherein, as the top of the diametrically opposing lobes engage each chamfer, each chamfer rides along its respective lobe, biasing the legs toward each other until the chamfer end is forced inward of the lobe.

12. The method according to claim 11, further comprising the step of:
(d) forcing the drill guide further downward and engaging the groove with the lobes, thereby allowing the legs to bias outwardly, securing the legs into the plate opening.

13. The method according to claim 12, further comprising the step of:
(e) releasing the guide.

14. The method according to claim 13, further comprising the step of:
(f) removing the drill guide by pinching the distal end of the guide legs inward toward each other until the groove is clear of the lobes; and
(g) lifting the drill guide upward out of the plate.

15. The method according to claim 13, further comprising the step of:
(f) angling the proximal ends of the legs until the groove snaps out of the lobes; and
(g) lifting the drill guide away from the plate.
